(19) United States
(12) Patent Application Publication
(10) Pub. No.: US 2004/0077530 A1
(43) Pub. Date: Apr. 22, 2004

(54) Composition for reducing caloric intake

(76) Inventor: Robert Portman, Fair Haven, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/274,071

(22) Filed: Oct. 18, 2002

(51) Int. Cl. A61K 38/17

(52) U.S. Cl. 514/8

(57) ABSTRACT

A composition for reducing caloric intake by stimulating the release of cholecystokinin following ingestion comprising glycomacropeptide and suitable excipients for ingestion in the substantial absence of other agents known to stimulate the release of cholecystokinin following ingestion. The subject compositions may additionally contain another agent that reduces caloric intake by a mode of action other than stimulating the release of cholecystokinin. The invention further comprises a method of achieving a reduction of caloric intake by causing an individual in need thereof to ingest an effective amount of the disclosed compositions with a meal or up to about ten minutes prior to a meal. An effective amount of the subject compositions to be consumed prior to or with a meal typically will provide from about 0.2 to about 3 grams of pure glycomacropeptide.

### FIELD OF INVENTION

The present invention relates to a nutritional composition for reducing caloric intake.

### BACKGROUND OF THE INVENTION

Development of safe and effective agents to help obese individuals lose weight has presented a major challenge to researchers. A significant increase in obesity among adults in the United States, and other countries as well, over the last twenty years has magnified the need to find safe and effective agents. Much of the research on finding a means to control and reduce obesity has focused on the control of appetite. The benefits of an agent that can help an individual consume less food and, as a result, fewer calories are obvious. A basic approach to accomplish this goal is to stimulate satiety, i.e. the feeling of fullness beyond satisfaction following ingestion of food. Research has shown that stimulating the mechanism of satiety generally results in the individual stopping eating sooner as opposed to changing his or her eating habits.

Satiety research has focused on three basis areas of physiology, i.e. those involving the appetite centers in the brain; the movement of food through the stomach; and the activation of peptides which are involved in the process of satiety itself. Because the brain plays an essential role in the control of appetite, researchers have studied various neurotransmitters, specifically, serotonin, dopamine and nor-epinephrine. A number of prescription and over-the-counter products have been developed that influence these neurotransmitters, thereby reducing appetite. However, reducing appetite by impacting brain neurotransmitters has been shown to have a number of drawbacks including loss of efficacy of the active pharmaceutical over time. More significant problems associated with agents that influence brain neurotransmitters, however, are side effects, particularly those associated with the activity of these agents on the central nervous system. Such side effects frequently include one or more of jitteriness, anxiety, and stimulation of the cardiovascular system.

The second approach to the problem of controlling obesity has focused on mechanisms of slowing gastric emptying, thereby creating and extending the feeling of fullness. This approach utilizes the administration of insoluble fibers, which act to retard the movement of food through the gastrointestinal tract. The disadvantage with the use of fiber, however, is that the quantities required to produce the desired effect create an unpalatable diet as well as numerous gastrointestinal side effects including bloating, flatulence and diarrhea.

The third approach has investigated means of activating the body's satiety mechanism. When food is consumed, various peptides are released that can stimulate satiety. The most important of these peptides appears to be cholecystokinin (CCK), a gastric peptide, which has been shown to be an important controller of satiety in humans. The release of CCK following ingestion of food produces several satiety effects, including slowing of gastric emptying and activation of receptors in the brain that, in turn, control appetite. Studies have shown that cholecystokinin is also extremely effective in extending satiety following ingestion of a meal. Although CCK has been shown to be effective in extending satiety and reducing food intake, it suffers the major disadvantage that it is inactivated by gastric enzymes. Hence, to be effective it must be administered intravenously. This has virtually defeated its potential use as a weight-loss agent.

In view of the fact that CCK is not effective upon oral administration, research has turned to studying means of stimulating the body to release it, thereby enhancing and extending satiety. It has been demonstrated that a number of nutritive agents, including protein, fat (particularly long chain fatty acids) and calcium, can stimulate the release of cholecystokinin. U.S. Pat. No. 4,491,578 discloses the oral administration of a trypsin inhibitor to enhance satiety by stimulating the release of cholecystokinin. The trypsin inhibitor is postulated to act by inhibiting the negative feedback signal for cholecystokinin secretion. In this fashion, the trypsin inhibitor sustains levels of CCK, thereby extending satiety. A proteinase inhibitor extracted from potatoes has likewise been shown to stimulate the release of cholecystokinin.

U.S. Pat. No. 6,207,638 teaches that a nutritional composition comprising protein, glycomacropeptide, C₁₂₋₁₈ fatty acids, calcium and both soluble and insoluble fibers can extend satiety by stimulating the release of CCK and, thereby, reduce food consumption. The preferred compositions disclosed in this patent contain about 80 calories. The disclosed compositions may be consumed prior to meals as well as included in specific foods to extend satiety thereof.

It is considered essential that individuals trying to lose weight reduce their caloric intake. Typically, the total calories in a caloric restricted diet will range between 1200-1500 per day. The compositions described in the '638 patent, although effective, if taken prior to each meal, represent a caloric contribution of about 240 calories or up to about 16% of the caloric daily requirements of a reduced caloric diet as noted above. In addition, the long chain fatty acid component of the disclosed compositions might not be ideal for certain individuals, such as those who, because they have high cholesterol, must be on a fat restricted diet, and the fiber component may produce food allergies in certain individuals, as well as fiber-associated side effects such as flatulence, bloating and GI distress.

Nutritional compositions that effectively stimulate the release of CCK in the body thereby aiding the consumer in a weight loss program are disclosed in the '638 patent. It has now unexpectedly been found in accordance with the present invention that a composition possessing advantageous properties can be formulated from a single component that stimulates the release of CCK in the body.

### SUMMARY OF THE INVENTION

The present invention provides for nutritional composition in a powder form for reducing caloric intake containing from about 30% to 90% by weight of glycomacropeptide as the only agent for the stimulation of CCK. The present invention further includes compositions that contain glycomacropeptide in combination with anti-obesity agents that are effective via mechanisms of action distinct from stimulating the release of CCK, and methods of causing reduced caloric intake utilizing such compositions. The compositions may be consumed as a powder, by addition thereof to food or a liquid, or in a tablet or capsule that can be ingested or added to food or a liquid.

### DETAILED DESCRIPTION OF THE INVENTION

In accordance with the present invention, it has been found that glycomacropeptide, consumed prior to a meal can reduce caloric intake more effectively than a composition containing glycomacropeptide in combination with other agents known to stimulate the production of CCK in the body. Caseinmacropeptide (CMP) or glycomacropeptide (GMP), which is fully-glycosylated CMP (also called kappa casein glycomacropeptide), is the first hydrolysis product resulting from the action of gastric proteinases on kappa casein. Glycomacropeptide has been shown to be involved in non-specific immunity and research thereon has been reported in the literature. Glycomacropeptides, i.e. glycosilated caseinmacropeptide, are utilized in the practice of the present invention.

Widely differing degrees of glycosylation of kappa casein macropeptide (CMP) exist in whey and whey products, ranging as noted, from GMP to non-glycosylated CMP. It is available commercially as part of whey protein in amounts ranging from 10-90% purity by weight. Ideally, the glycomacropeptide utilized in the practice of the present invention is substantially 100% pure. However, until such time as the economies of its use warrant the expense of developing a glycomacropeptide product substantially 100% pure, it is be added to the preparations of the present invention as compositions containing from about 30% to about 60% by weight and higher, e.g. up to and above 90% by weight.

The commercially available glycomacropeptide preparations are composed of pure glycomacropeptide and other whey proteins in inverse ratio to the purity of the preparation. The presence of such proteins has no deleterious effect on the intended use of the subject compositions since they are known to have some stimulant activity on CCK production. However, it has been found in accordance with the present invention that glycomacropeptide is more efficient when administered in the substantial absence of other agents that stimulate CCK production in the body, hence such other proteins, to the extent they are present, are considered incidental and not required for the performance of the subject compositions. In accordance with the present invention, replacement of such proteins with a like amount of glycomacropeptide as commercial preparations reach higher purity would enhance the performance of the subject compositions.

In addition to the unexpected efficacy of the compositions of the present invention, the fact that they utilize only highly purified glycomacropeptide as an active ingredient provides a number of other significant advantages in comparison to other similar compositions known to those of ordinary skill in the art. A first consideration is ease or formulation and manufacture. Because the subject compositions preferably contain only a single active ingredient, they are more convenient and less expensive to manufacture, have a significantly smaller bulk than prior compositions that contain four or five active ingredients, and are more readily stabilized, packaged, stored and the like. In addition, glycomacropeptide can be instantized by agglomeration with the addition of a natural emulsifier, preferably lecithin, during the agglomeration process. The resulting agglomerates are easily dissolved in both liquids and semisolid foods such as yogurt and puddings. In formulations that may be taken with a meal, the subject compositions are more readily combinable with foods to increase their satiety without adversely impacting their texture and taste.

Compositions prepared in accordance with the present invention are advantageous, for example, in that they may be formulated into tablets or filled into capsules, a decided convenience to the consumer. The tablets may be friable so that they can be broken and added to a liquid, or chewed since the taste of glycomacropeptide can be masked with conventional artificial sweetening and flavoring agents. Also, since there is only a single active principal for stimulating the release of CCK in the body, the compositions of the present invention may be formulated with other agents that act to reduce appetite or cause weight loss by mechanisms other than and distinct from stimulating the release of CCK in the body. It should be evident to those of ordinary skill in the art that agents that accomplish the same result in the body via different mechanisms of action often compliment each other's activity and may even exert synergistic activity.

Appetite suppression agents that may be combined with glycomacropeptide in the compositions of the present invention may include stimulants, such as caffeine and Ephedrine, swympathomimetic agents, such as phentermine or Sibutramine, marketed under the trademark Meridia by Knoll Pharmaceuticals, lipase inhibitors, such as Orlistat, marketed under the trademark Zenical by Roche Laboratories, Inc., and the like. There is advantage in the combination of such agents with glycomacropeptide in the compositions of the present invention in that such agents may be utilized in less than their normal dosage, thereby reducing the incidence of side effects usually associated with their use as discussed above. While the amount of such agents to be formulated into the subject compositions may vary, in general, between 40% and 70% of their usual dosage is contemplated, depending on factors such as compatibility, the amount of active material to be incorporated into the subject compositions and the like.

A marked advantage of the compositions of the present invention in comparison to prior compositions containing glycomacropeptide is that the subject compositions add as little as 12 calories, more typically about 20 calories, to a meal. This is significant for individuals on a restricted diet as it permits more flexibility and variety in the diet within the constraints of a given diet program. This is a very important consideration because it has been shown that a primary reason why individuals give up on restricted diet programs is the lack of variety in the choice of permitted foods. In contrast, the compositions of the present invention permit the individual to consume a wide variety of foods because they are simply consuming less.

Another advantage of the subject composition is that they can be utilized on a maintenance program for individuals who have lost weight and do not want to regain it. This is due to the fact that the subject compositions do not contribute many calories to the daily intake and they permit the individual to eat a variety of foods. An individual who has lost weight and does not wish to regain it might consume the subject compositions with only one or two meals daily, preferably with the biggest meal of the day, or with the evening meal as it has been shown that fewer people exercise after the evening meal than with the other meals of the day.

The subject compositions are also advantageous in comparison with such prior compositions in that they do not contain fat and/or fiber thereby eliminating the drawbacks and potential reactions to such substances described above. In addition to the foregoing advantages, the subject compositions containing glycomacropeptide as the only active principal that stimulates the release of CCK in the body are highly efficacious in that, used in a regular regimen, patients generally experience a reduction in caloric intake of up to about twelve percent.

The subject compositions are comprised of glycomacropeptide, conventional excipients, binders, flavoring agents, artificial sweetening agents, pigments and the like. Conventional pharmaceutical diluents or excipients may include one or more of emulsifiers, fillers, binders, lubricants, binders, compression aids, wetting agents and the like. Tablets prepared from the subject formulations may additionally contain conventional disintegrating agents. For incorporation into a liquid to form a drink, the compositions may contain emulsifiers, including but not limited to lecithin and phosphatidyl choline derivatives, acacia, or veegum and one or more surfactants, particularly non-ionic surface active agents, for example the Tween series.

The composition may likewise include coloring agents, or pigments, such as FD&C or D&C approved lakes and dyes, iron oxide and titanium dioxide, sweeteners such as aspartame, sodium cyclamate and sodium saccharinate, and non-natural sugars, such as dextrose, sucrose, fructose, mannitol and xylitol. The flavoring component may include, without intended limitation, water soluble, natural or artificial extracts of apple, banana, cherry, cinnamon, cranberry, grape, honeydew, honey, kiwi, lemon, lime, orange, peach, peppermint, pineapple, raspberry tangerine, watermelon, wild cherry and the like.

The subject compositions may be administered as a powder formulation, in bulk or in unit dose/sachets for patient ease to be consumed directly or, preferably mixed with a food or foods, or a suitable liquid such as water or a juice. It is also contemplated that the composition may be formulated as a tablet that may be chewed, swallowed or that will rapidly disintegrate in a suitable liquid to form a drink.

The subject compositions comprise from about 30% to about 90% by weight of glycomacropeptide with the remainder being excipients as described above in the substantial absence of other agents know to stimulate the release of CCK in the body. The percent by weight of glycomacropeptide given above represents pure glycomacropeptide in a commercial preparation containing it. Hence, the lower percentages given would incorporate commercial forms containing less pure glycomacropeptide, e.g. 30% purity by weight, whereas the higher percentages would incorporate commercial form containing a more pure glycomacropeptide, e.g. 90% and above. It will be appreciated that preparations containing a lesser amount of glycomacropeptide than taught in accordance with the present invention could be utilized, but would not offer the practical benefits of the subject compositions.

The balance of the compositions of the subject invention is comprised of the excipients as detailed above. The amount and selection of the type of excipients would depend on the desired final form of the preparation. For example, a tablet would contain a disintegrating agent, a chewable tablet would contain additional flavoring and sweetening components, whereas a tablet or powder to be added to a liquid would contain at least one of emulsifiers and surfactants that would be absent, or present in a reduced quantity, in a powder to be incorporated into a food.

The amount and relative proportion of the excipient materials are considered to be within the purview of a person skilled in the arts of pharmaceutical compounding or food product formulating.

As examples of foods to which the compositions of the present invention may be added for ingestion prior to or as part of a meal, there is considered yogurt, puddings, gelatin dessert, apple sauce, cottage cheese, cereal, bread, and candy or nutritive bars. The subject compositions may also be added to liquids including but not limited to water, apple juice, orange juice, grape juice, grapefruit juice, cranberry juice, coffee, tea, milk, milkshakes, broth, and soup consommé.

As stated above, it is a significant advantage of the compositions of the present invention that they can add as low as only twelve, more typically about twenty calories to a meal. This means that, in a daily regimen of even as many as four dosages, the subject compositions will still add less than one hundred calories to the diet of the individual in need thereof. Although the dosage regimen may be varied depending on the needs of the individual, the amount of the compositions of the present invention to be administered to a patient in need thereof in a single dosage is typically sufficient to provide from about 0.2 to 3 grams of pure glycomacropeptide. Preferably, a single dosage will provide from about 1 to 2 grams of pure glycomacropeptide. The subject compositions are to be administered with a meal or just prior there, i.e. within about 10 minutes, preferably about 5 minutes, prior to the meal.

The following examples further illustrate the present invention, it being understood that they are in no way intended as limiting the scope thereof.

### EXAMPLE 1

Twenty obese subjects (BMI 25-31) were administered one of the following preparations, each of which was rated at 80 calories:

1) A carbohydrate placebo preparation consisting of maltodextrin and flavoring;

2) A preparation containing oleic acid (2.4 g), whey protein isolate (2.5 g), glycomacropeptide (0.5 g), potato flour (3.0 g), glucomannan (1.0 g), guar gum (0.76), calcium lactate (0.64 g), alfalfa (0.1 g) plus flavorings and artificial sweetener; and

3) A preparation containing glycomacropeptide (1.50 g pure material content) plus flavorings and artificial sweetener with the substantial absence of other agents known to cause release of CCK in the body.

The study was a crossover, randomized single blind study. Subjects meeting the study criteria were instructed not to eat or drink anything, except water, from 10:00 p.m. on the night before the study and not to consume any alcohol in the preceding 24 hours.

Each study day, the subjects arrived at the test facility at 8:30 a.m., were weighed and consumed a breakfast totaling 200 calories. The subjects were free to consume as much water as they wished until one hour before the pre-meal beverage was consumed.

Four hours following breakfast, the subjects were provided with eight ounces of one of the three pre-meal beverages. Fifteen minutes after the pre-meal beverage was consumed, the subjects were provided with a pasta dish lunch and instructed to consume as much food as they desired within a 25 minute period. The amount of food consumed by each subject was measured and recorded.

Following lunch, the subjects were asked to complete VAS (Visual Analog Scale) assessments for hunger, fullness, thirst and how much food they want to eat. VAS assessments were recorded at one hour, two hours and three hours after lunch. The caloric consumption data was analyzed using Analysis Of Variance (ANOVA) and t-test comparing the three treatments.

In addition, VAS data at each time period was analyzed using ANOVA.

It was found that the subjects consuming preparation 3) containing glycomacropeptide decreased food intake by 12.5% as compared to 7.5% for preparation 2) which contained glycomacropeptide in combination with other agents known to stimulate CCK release in the body, both verses the control preparation 1), a 38% improvement in efficacy. The difference between the preparation of the present and preparation 2) was considered statistically significant.

Moreover, the VAS values for the preparation of the present invention, preparation 3), were essentially the same for those for preparation 2), yet the subjects consumed less food with preparation 3) and, therefore, fewer calories. The unexpected advantages of the subject compositions are readily apparent from the results of this study.

### EXAMPLE 2

A total of 6.5 grams of glycomacropeptide (61% pure obtained from Apollo, Ottawa, Canada) and 50 mg of soy lecithin, a surfactant/emulsifier available from Central Soya, Fort Wayne, Ind., were mixed until homogeneous and added with stirring to 16 oz of a commercially available, artificially sweetened ice tea obtained from Snapple Beverage Corporation, White Plains, N.Y.). Eight ounces of the resulting refreshing, pleasant-tasting beverage contained 2 grams of pure glycomacropeptide.

### EXAMPLE 3

A total of 13.0 grams of glycomacropeptide (61% pure obtained from Apollo, Ottawa, Canada) and 100 mg of soy lecithin were mixed until homogeneous and added with stirring to 32 oz of a commercially available orange juice (Tropicana, Bradenton, Fla.). Eight ounces of the resulting solution contained 2 grams of pure glycomacropeptide.

### EXAMPLE 4

A total of 3.25 grams of glycomacropeptide (61% pure obtained from Apollo, Ottawa, Canada) and 50 mg of soy lecithin were mixed until homogeneous and then blended with 8 oz of plain, low-fat yogurt (The Dannon Company, Inc., Tarrytown, N.Y.) until homogeneous. The resulting mixture contained 2 grams of pure glycomacropeptide.

### EXAMPLE 5

A total of 3.25 grams of glycomacropeptide (61% pure obtained from Apollo, Ottawa, Canada) and 50 mg of soy lecithin were mixed until homogeneous and then blended with 8 oz of Jell-O® pudding snacks (Kraft Foods Inc., Rye Brook, N.Y.) until homogeneous. The resulting mixture contained 2 grams of pure glycomacropeptide.

What is claimed:

1. A composition for reducing caloric intake comprising glycomacropeptide that stimulates the release of cholecystokinin following ingestion and suitable excipients for ingestion in the substantial absence of other agents known to stimulate the release of cholecystokinin following ingestion.

2. A composition in accordance with claim 1, wherein said composition is in powder form and further comprises excipients suitable for the ingestion of said powder.

3. A composition in accordance with claim 1, wherein said composition is in powder form and further comprises excipients suitable for the addition of said powder to food.

4. A composition in accordance with claim 1, wherein said composition is in powder form and further comprises excipients suitable for the addition of said powder to a liquid to form a drink.

5. A composition in accordance with claim 1, wherein said composition is in the form of a tablet that may be chewed or swallowed, and said excipients are suitable for the formulation of such tablets.

6. A composition in accordance with claim 1, wherein said composition comprises from about 30% to about 90% by weight of pure glycomacropeptide.

7. A composition in accordance with claim 1, wherein said glycomacropeptide is present as a purified whey preparation containing at least thirty percent by weight of pure glycomacropeptide.

8. A composition in accordance with claim 7, wherein said whey preparation contains at least about sixty percent by weight of pure glycomacropeptide.

9. A composition in accordance with claim 1 additionally containing an agent that achieves caloric reduction by a mode of action other than the stimulation of the release of cholecystokinin following ingestion.

10. A composition in accordance with claim 9, wherein said agent is a stimulant.

11. A composition in accordance with claim 10, wherein said stimulant is caffeine or ephedrine.

12. A composition in accordance with claim 9, wherein said agent is a sympathomimetic agent.

13. A composition in accordance with claim 9, wherein said agent is a lipase inhibitor.

14. A method of reducing the caloric intake in an individual in need thereof comprising causing said individual to ingest with a meal or up to about ten minutes prior thereto an effective amount of a composition that stimulates the release of cholecystokinin following ingestion comprising glycomacropeptide and suitable excipients for ingestion in the substantial absence of other agents known to stimulate the release of cholecystokinin following ingestion.

15. A method in accordance with claim 14, wherein a sufficient amount of said composition is ingested to provide from about 0.2 to about 3 grams of pure glycomacropeptide.

16. A method in accordance with claim 15, wherein a sufficient amount of said composition is ingested to provide from about 1 to about 2 grams of pure glycomacropeptide.

17. A method in accordance with claim 14, wherein said composition is in powder form that is added to a food prior to ingestion.

18. A method in accordance with claim 14, wherein said composition is in powder form that is added to a liquid to form a drink prior to ingestion.

19. A method in accordance with claim 18, wherein said liquid is selected from the group consisting of water, apple juice, orange juice, grape juice, grapefruit juice, cranberry juice, coffee, tea, milk, milkshakes, broth, and soup consommé.

20. A method in accordance with claim 14, wherein said composition additionally contains an agent that achieves caloric reduction by a mode of action other than the stimulation of the release of cholecystokinin following ingestion.

21. A method in accordance with claim 20, wherein said agent is a stimulant.

22. A method in accordance with claim 21, wherein said stimulant is caffeine or ephedrine.

23. A method in accordance with claim 20, wherein said agent is a sympathomimetic agent.

24. A method in accordance with claim 20, wherein said agent is a lipase inhibitor.
